(19) United States
(12) Patent Application Publication
(10) Pub. No.: US 2024/0403670 A1
(43) Pub. Date: Dec. 5, 2024

(54) METHODS AND SYSTEMS FOR SELECTING ACTIONS FROM A SET OF ACTIONS TO BE PERFORMED IN AN ENVIRONMENT AFFECTED BY DELAYS

(71) Applicant: THE ROYAL INSTITUTION FOR THE ADVANCEMENT OF LEARNING/MCGILL UNIVERSITY, Montreal (CA)

(72) Inventors: Sebastian PILARSKI, Montreal (CA); Slawomir PILARSKI, Vancouver, WA (US); Daniel VARRO, Westmount (CA)

(21) Appl. No.: 18/294,356

(22) PCT Filed: Aug. 5, 2022
(86) PCT No.: PCT/CA2022/051196

§ 371 Date: Feb. 1, 2024

Related U.S. Application Data
(60) Provisional application No. 63/229,711, filed on Aug. 5, 2021.

(51) Int. Cl. G06N 7/01; G06F 17/18; G06N 3/092; G06Q 50/12; G16H 20/10

(52) U.S. Cl. CPC G06N 7/01; G06F 17/18; G06N 3/092; G06Q 50/12; G16H 20/10

(57) ABSTRACT

A method of selecting an action from a plurality of actions to be performed in an environment comprises maintaining. for each action, count data indicative of a number of times the action has been performed and a difference between the number of times and a number of observed resulting rewards for the action, each reward being a numeric value that measures an outcome of a given action, determining. from the count data and a bandit score provided by a bandit model. an expected score for each action, the bandit score provided by the bandit model for a given history of performed actions and observed rewards. and the expected score determined by determining an expected value of the bandit score given a likelihood of some of the actions having unobserved pending rewards. and selecting the action from the actions and based on the expected score for each action.

### CROSS-REFERENCE TO RELATED APPLICATIONS

The present application claims the benefit of U.S. Provisional Patent Application No. 63/229,711 filed on Aug. 5, 2021, the contents of which are hereby incorporated by reference in their entirety.

### TECHNICAL FIELD

The present disclosure is directed to decision making systems based on reinforcement learning models.

### BACKGROUND OF THE ART

Multi-armed bandits are a reinforcement learning model used to study a variety of choice optimization problems. It assumes that decisions are sequential and at each time one of a number of options is selected. Its name reflects the quandary of a casino gambler, or a player, who attempts to maximize his total winnings, or a cumulative reward, when facing a row of slot machines called 1-arm bandits. The model assumes that each arm, when pulled, produces a random reward according to its own probability distribution, which is unknown to the player. For example, a Bernoulli bandit is a multi-armed bandit used to model processes where the outcome of a decision is strictly binary: success/failure, yes/no, or 1/0; each arm is characterized by its own probability of success. Multi-armed bandits can be used, for example, in pre-clinical and clinical trials, telecommunications, portfolio/inventory management, A/B testing (e.g., news headline selection, click feedback), and dynamic pricing. If a reward associated with an arm pull is unknown until d subsequent decisions have been taken, the reward has a delay of d decisions. Delayed rewards are sometimes referred to as “delayed feedback”. Many problems exhibit delays in information response after decisions are made. Most bandit algorithms are not designed to deal with delays and their performance rapidly decreases as the delay between a decision and its outcome grows. Well-known bandit algorithms are ill-equipped to deal with still unknown (delayed) decision results, which may translate into significant losses, e.g., the number of unsuccessfully treated patients in medical applications, decreased bandwidth in telecommunications, or resource waste in inventory management.

As such, there is room for improvement.

### SUMMARY

In accordance with a broad aspect, there is provided a method of selecting at least one action from a plurality of actions to be performed in an environment. The method comprises maintaining, for each action from the plurality of actions, count data indicative of a number of times the action has been performed and a difference between the number of times the action has been performed and a number of observed resulting rewards for the action, each reward being a numeric value that measures an outcome of a given action, determining, from the count data and a bandit score provided by a bandit model, an expected score for each action from the plurality of actions, the bandit score provided by the bandit model for a given history of performed actions and observed rewards, and the expected score determined by determining an expected value of the bandit score given a likelihood of some of the plurality of actions having unobserved pending rewards, and selecting, from the plurality of actions and based on the expected score for each action, the at least one action to be performed in the environment.

In some embodiments, selecting the at least one action comprises selecting a resource allocation to implement in a telecommunications environment.

In some embodiments, selecting the at least one action comprises selecting at least one of a drug to administer, a treatment to provide, medical equipment to use, and device options to set in a clinical trial or a pre-clinical trial environment.

In some embodiments, selecting the at least one action comprises selecting an experimental option from a plurality of experimental options to evaluate in an experimental environment.

In some embodiments, selecting the at least one action comprises selecting a channel from a plurality of channels to use in an Internet-of-Things (IoT) environment.

In some embodiments, selecting the at least one action comprises selecting at least one of a price at which to set one or more products, an amount of the one or more products to order, and a time at which to the order one or more products in a food retail environment.

In some embodiments, the method further comprises receiving an indication that a reward was observed in response to a selected one of the plurality of actions being performed, and in response, updating the count data.

In some embodiments, maintaining the count data comprises maintaining a count of the difference between the number of times the action has been performed and the number of resulting rewards have been observed for a given action, the count being a windowed count that counts how many times a reward has been observed for a given action in response to the action being performed during a recent time window that includes a fixed number of most recent time steps.

In some embodiments, the bandit score is determined using a Whittle index.

In some embodiments, the bandit score is determined using an infinite time horizon algorithm.

In some embodiments, the expected score for each action is determined by determining all discrete possible reward configurations using the count data and determining probabilities of the possible reward configurations using state transition probabilities, and multiplying a probability of each possible reward configuration by the bandit score of each action of the given history of performed actions.

In some embodiments, the bandit score is determined using one of probabilistic sampling and Monte Carlo simulation. A probabilistic sample is defined as simulating a sequence of actions for missing rewards in the count data, updating a prior expected action reward distribution after each simulated action, and determining the bandit score using simulated rewards when no missing rewards remain, the probabilistic sampling using a number of probabilistic samples to create an estimate of the expected score.

In some embodiments, the rewards measure discrete outcomes.

In some embodiments, the rewards measure an uncountable set of outcomes defining a continuous probability distribution.

In some embodiments, the expected score is an average expected score based on a number N of samples, and further wherein selecting the at least one action to be performed in the environment comprises selecting a highest average expected score.

In accordance with another broad aspect, there is provided a system comprising one or more computers and one or more storage devices storing instructions that when executed by the one or more computers cause the one or more computers to perform operations for selecting at least one action from a plurality of actions to be performed in an environment. The operations comprise maintaining, for each action from the plurality of actions, count data indicative of a number of times the action has been performed and a difference between the number of times the action has been performed and a number of observed resulting rewards for the action, each reward being a numeric value that measures an outcome of a given action, determining, from the count data and a bandit score provided by a bandit model, an expected score for each action from the plurality of actions, the bandit score provided by the bandit model for a given history of performed actions and observed rewards, and the expected score determined by determining an expected value of the bandit score given a likelihood of some of the plurality of actions having unobserved pending rewards, and selecting, from the plurality of actions and based on the expected score for each action, the at least one action to be performed in the environment.

In some embodiments, the operations further comprise receiving an indication that a reward was observed in response to a selected one of the plurality of actions being performed, and, in response, updating the count data.

In some embodiments, maintaining the count data comprises maintaining a count of the difference between the number of times the action has been performed and the number of resulting rewards have been observed for a given action, the count being a windowed count that counts how many times a reward has been observed for a given action in response to the action being performed during a recent time window that includes a fixed number of most recent time steps.

In some embodiments, the expected score for each action is determined by determining all discrete possible reward configurations using the count data and determining probabilities of the possible reward configurations using state transition probabilities, and multiplying a probability of each possible reward configuration by the bandit score of each action of the given history of performed actions.

In some embodiments, the bandit score is determined using one of probabilistic sampling and Monte Carlo simulation. A probabilistic sample is defined as simulating a sequence of actions for missing rewards in the count data, updating a prior expected action reward distribution after each simulated action, and determining the bandit score using simulated rewards when no missing rewards remain, the probabilistic sampling using a number of probabilistic samples to create an estimate of the expected score.

### BRIEF DESCRIPTION OF THE DRAWINGS

Reference is now made to the accompanying figures in which:

FIG. 1 is a graph of probability density functions of the Beta distribution.

FIG. 2 is a set of graphs of clinical trial simulation results for a 2-arm Bernoulli bandit with arm probabilities of success 0.77 and 0.22. UCBT: no delay vs. delay of 24.

FIG. 3 is a set of graphs showing the regret of common algorithms. No delay vs. delay of 24.

FIG. 4 is the probabilistic analysis of reward outcomes. 1-arm, three unknown rewards.

FIG. 5 is the optimal policy regret for various delays.

FIG. 6 is the excess regret caused by delay.

FIG. 7 is a set of graphs showing the reduction of regret for common algorithms using the method presented herein, delay of 24.

FIG. 8 is a set of graphs showing the reduction of regret as a function of delay for a time horizon of 200 using the method presented herein with Whittle Index scoring.

FIG. 9 is a set of graphs showing the reduction of regret vs. the number of arms for a time horizon of 200 using the method presented herein with Whittle Index scoring.

FIG. 10 is a set of graphs showing the regret reduction for various algorithms, delays and numbers of arms using the method presented herein.

FIG. 11 is a set of graphs showing the regret performance for UCB1 and UCBT algorithms for 2-arms, 10-arms, and 30-arms in the presence of different standard deviations and no (0) delay and delays of 50, also showing the regret performance of these algorithms when combined with the method presented herein.

FIG. 12 is a set of graphs showing the effect of the number of arms and delay.

FIG. 13 illustrates sets of graphs showing the improvement in regret as a function of arms and delays using the method presented herein.

FIG. 14 is a schematic diagram of an example computing system.

### DETAILED DESCRIPTION

The present disclosure is directed to decision making systems. Choices must be made when playing games, buying products, routing telecommunications, setting prices, and when treating medical patients. The greater the complexity of the outcome, the more difficult finding an optimal strategy must be. Traditionally, in clinical trials and A/B testing, determining a superior selection choice was accomplished by allocating an equal number of samples into each option and seeing which option performed better on average. However, in marketing or medicine, such a methodology can greatly decrease the number of purchasing consumers or treated patients during testing. Likewise, with small testing populations, this may not be the best strategy for maximizing information gain or optimizing the outcome.

At the beginning of testing (e.g., news headline testing or clinical trials), the efficacy of each option is unknown. During testing, each option is “measured” against others. Some test subjects will receive a superior option (e.g., more click-inducing headline or better medical treatment than others). This is an inevitable price of knowledge acquisition. Achieving the greatest number of article reads or successfully treating the largest number of patients is of the utmost priority.

In many situations the process of selecting options is sequential. Such situations are studied using a reinforcement learning model referred to as a multi-armed bandit. Often, the sequence length is finite.

A multi-armed bandit is the problem of a player (or a gambler), who facing a row of different one-arm slot machines attempts to maximize the total gain, or cumulative reward, when he or she is allowed to pull only one arm at a time. The player does not know expected payoffs, so a successful strategy has to effectively balance exploration and exploitation. Some algorithms assume that the game continues forever, while others consider finite-time horizons, i.e., situations where the player has a fixed total number of arm pulls.

A large number of real-life decision-making scenarios can be modeled using multi-armed bandits. Reward outcomes can be modeled as discrete (or countable) outcomes (e.g., true/false, yes/no, success/failure, good/neutral/bad), or numerical values (revenue, number of clicks, throughput, etc.). In multi-armed bandits, reward outcomes are characterized by their reward distributions. Conceptually, given an arm's pre-defined distribution (referred to herein as a “prior”) and a time horizon (number of sequential decisions) it is possible to find the best strategy or deterministic optimal policy (OPT). However, computing such policies for all but small examples in many forms of multi-armed bandits is currently regarded as impossible and thus, in practice, suboptimal algorithms are used.

As an example, the Bernoulli multi-armed bandit is advocated as an alternative to traditional randomized clinical trials where patients are divided into similar size cohorts and each cohort receives a different drug or treatment. Bernoulli bandit algorithms optimize the expected number of patients successfully treated during a trial. Each arm corresponds to a unique treatment and the arm probability is the likelihood that a patient would achieve a success criterion, e.g., reaching a given level of antibodies. An algorithm selects a treatment for each patient based on observed successes and failures in previous patients. A trial vaccine could be compared to a placebo, another vaccine, or various levels of dosage. Such trials may range from a few hundred to thousands of participants and generally compare only two or three treatment options.

Computing optimal policies (OPTs) for multi-armed bandits is commonly considered practically infeasible due to the computational complexity of the problem. As a consequence, suboptimal algorithms are used in practice. Existing empirical data regarding the level of suboptimality of such algorithms is very limited in scope from a practical point of view. Uniform priors are typically assumed which is usually not satisfied in real-world scenarios. Consequently, there is a need to explore new computation methods to produce OPTs for previously infeasible time horizons, number of arms, and various priors. It is also desirable to assess the level of suboptimality for a wide range of existing bandit algorithms.

As used herein, the term “count data” will refer to data that is maintained by the methods and systems described herein and that is indicative of a number of times an action has been performed and of a difference between the number of times the action has been performed and a number of observed resulting rewards for the action. As will be described further below, from the count data and a bandit score provided by a bandit model, an expected score for each action from a plurality of actions is determined. The bandit score is provided by the bandit model for a given history of performed actions and observed rewards, and the expected score is determined by determining an expected value of the bandit score given a likelihood of some of the plurality of actions having unobserved pending rewards. At least one action to be performed in an environment is then selected from the plurality of actions, based on the expected score for each action.

In the present disclosure, notions of probability theory such as expected value, (arithmetic) mean, standard deviation, and variance of a random variable are used. The standard notion of a cumulative mean is also used, which is the arithmetic mean of means over a period of time. Cumulative also applies to other random variables, not just the mean. The following common notations are used:
P[A] is the probability of an event A
E[X] denotes the expected value of a random variable X
σ denotes standard deviation of a random variable
σ² denotes variance of a random variable
σ\hat{ } denotes deviation of observed values
Z is the set of integers
argmax_{i=1, . . . , k}(g(i)) selects i with max value of g(i)
argmin_{i=1, . . . , k}(g(i)) selects i with min value of g(i)

A multi-armed bandit, or MAB, has some number of decision options/actions (arms) k and some finite or infinite time horizon H. At each discrete time (step) 0≤t<H, the player (or an algorithm) chooses one or more of the k arm options and receives a reward. Reward outcomes are drawn from corresponding reward distributions. A game or experiment consists of making decisions sequentially through all time steps to time horizon H.

In the present disclosure, when presenting or discussing bandit algorithms, the following symbols are used:
k is the number of arms
H is the time horizon (i.e. the number of pulls in a single game)
t is time, 0≤t≤H
nᵢ is the number of times a reward for arm i was observed
uᵢ is the number of times arm i was pulled without an accompanying reward observed
sᵢ is the number of times arm i produced a success in a Bernoulli bandit
fᵢ is the number of times arm i produced a failure in a Bernoulli bandit
μᵢ is the expected value of reward for arm i
μ\hat{ }i is the observed mean reward for arm i

μᵢ, μ\hat{ }ᵢ, and nᵢ apply to a single game, i.e., drawing arm's probabilities and playing one game that consists of a sequence of single arm pulls.

Also, for a single game, at any time t, Σᵢ₌₁^{k}(nᵢ+uᵢ)=t. In a single game, nᵢ(t) and uᵢ(t) are functions of t. nᵢ(t)+uᵢ(t) denotes the number of times arm i has been pulled by time t. In a single Bernoulli bandit game, nᵢ=sᵢ+fᵢ.

For evaluation of decision-making methods in a controlled simulation environment, it is convenient to have a symbol for the arm which guarantees the highest expected reward outcome. This is referred to as the “best arm”, as follows: best arm=argmax_{i=1, . . . , k}(μᵢ). In the present disclosure, symbols associated with the best arm are labeled with a *.μ*=max_{i=1, . . . , k}(μᵢ) is the best expected reward and μ\hat{ }*=max_{i=1, . . . , k}(μ\hat{ }ᵢ) is the best observed mean reward. In a single game μ* is a constant, while μ\hat{ }*ᵢ is a random variable. Also, in the vast majority of published algorithms, uᵢ=0 (i.e., there is an assumption of no delays).

Computed over multiple games E[μ*] is an obvious upper bound on the mean reward of any player's algorithm at any time t. E[μ*] is a function of the distribution of arm success probabilities and the number of arms k.

A common way to compare the performance of different methods in multi-armed bandits is to examine their loss of opportunity (also referred to herein as “regret”), which experimentally is the mean of the difference between the best arm's success rate and the selected arm's success rate, computed over a number of simulation runs/games/experiments.

Formally, regret(t)=E[μ*−μ(t)] where μ(t) is the expected reward of the arm selected by the player at time t in a single game. μ* is a constant in a single game, but a random variable when multiple games are run. Minimization of cumulative regret is equivalent to maximizing cumulative reward.

For simulation-based experimental evaluation of methods, it is common to assign reward distributions to bandit arms. For example, in Bernoulli bandits, each experiment starts with assigning each arm its success probability. This probability is a random variable drawn from a probability distribution, i.e. a prior. In Bernoulli bandit simulations, the Beta distribution is used. The probability density function of the Beta distribution is given by the following equation:

$$\frac{{{x}^{α - 1} ( 1 - x )}^{β - 1}}{{\int }_{0}^{1} {{y}^{α - 1} ( 1 - y )}^{β - 1} dy} . \qquad ( 1 )$$

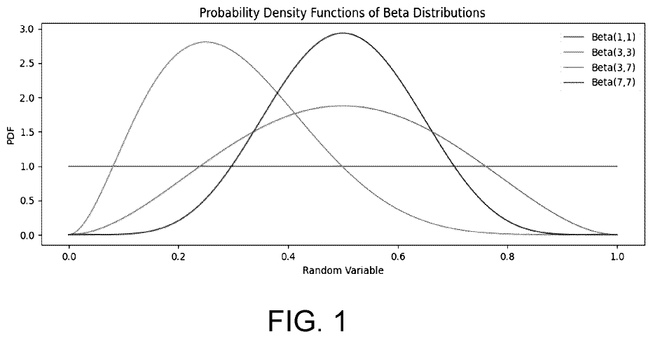

FIG. 1 presents plots of the Beta distribution for selected values of parameters α and β.

Property 1 (Conjugate Prior): Pulling an arm in a Bernoulli bandit changes its distribution from Beta(α,β) to Beta(α+1,β) if the outcome is a success, or to Beta(α,β+1) if the outcome is a failure. This property, together with equation (2) below, plays a vital role in computations used in some algorithms discussed in the present disclosure.

$$𝔼 ( Beta ( α , β ) ) = \frac{α}{α - β} \qquad ( 2 )$$

The Beta distribution's standard deviation is as follows:

$$σ ( Beta ( α , β ) ) = \sqrt{\frac{αβ}{{( α + β )}^{2} ( α + β + 1 )}} \qquad ( 3 )$$

There exist popular multi-armed bandit algorithms which attempt to optimize decision-making in multi-armed bandits. These include, but are not limited to, Thompson Sampling (TS), EXP4, UCB1-Tuned (UCBT), LinUCB, and Whittle Index (WI).

TS is a randomized algorithm presented herein the context of Bernoulli bandits. In essence, it generates random numbers according to each arms' Beta distribution, and picks the arm with the largest random number.

$$choose ( t ) = {arg max}_{i = 1 , … k} ( random ( Beta ( {α}_{i} ( t ) , {β}_{i} ( t ) ) ) ) \qquad ( 4 )$$

EXP4 is a contextual bandit algorithm. Chooses arm a by drawing it according to action probabilities q̅, which are computed using experts' recommendations.

$$choose ( t ) = arg {max}_{i = 1 , … k} ( 𝕀 ( a = i ) ) , where arm a \sim \overline{q} \qquad ( 5 )$$

The UCBT algorithm is an improved version of the UCB1 algorithm.

$$choose ( t ) = {arg max}_{i = 1 , … k} ( \hat{{μ}_{i}} ( t ) + c \sqrt{\frac{ln t}{{n}_{i} ( t )} min ( \frac{1}{4} , {V}_{i} ( t ) )} ) \qquad ( 6 )$$

$${V}_{i} ( t ) = {\hat{σ}}_{i}^{2} ( t ) + \sqrt{\frac{2 ln t}{{n}_{i} ( t )}}$$

where σ\hat{ }ᵢ² is the variance of the success rate, and c is a constant. The constant c controls the degree of exploration. In the present disclosure, c=1 for all experiments—the common default value used in literature.

The LinUCB algorithm is an adaptation of the UCB algorithm to contextual bandit scenarios:

$$choose ( t ) = arg {max}_{a = 1 , … , k} ( {\hat{θ}}_{a}^{T} {x}_{t , a} + α \sqrt{{x}_{t , a}^{T} {A}_{a}^{- 1} {x}_{t , a}} ) \qquad ( 7 )$$

where Aₐ is initialized as a d-dimensional identity matrix, and bₐ as a d-dimensional zero vector. \hat{θ}ₐ=Aₐ⁻¹bₐ and after each decision Aₐ_{ₜ} and Bₐ_{ₜ} are updated by Aₐ_{ₜ}=Aₐ_{ₜ}+x_{t,a}_{ₜ}x_{t,a}_{ₜ}^{T} and bₐ_{ₜ}=bₐ_{ₜ}+r_{t,a}_{ₜ}x_{t,a}_{ₜ}, respectively.

Whittle Index is a modification of Gittins Index, which was a milestone in developing Bernoulli bandit algorithms. It is modified for finite time horizons, as follows:

$$choose ( t ) = {arg max}_{i = 1 , … , k} ( {W}_{I} ( {s}_{i} ( t ) + {α}_{i} , {f}_{i} ( t ) + {β}_{i} , H - t ) ) \qquad ( 8 )$$

where WI is the Whittle index, H is the time horizon, sᵢ and fᵢ are the numbers of successes and failures observed for arm i, and Beta(αᵢ,βᵢ) is the distribution from which success probability of arm i was drawn.

It has been shown empirically that Whittle Index offers nearly-optimal regret for a variety of numbers of arms and their priors regardless of the time horizon.

Additionally, in some circumstances it is possible to compute the optimal policy which guarantees best expected decision-making performance.

In the context of Bernoulli bandits, there exists a statistically optimal deterministic policy (also referred to herein as “optimal policy” or OPT). It can be computed for a given number of arms and their respective Beta priors, as follows:

$$choose ( t ) = {O}_{{P}_{H}} ( ( {s}_{1} ( t ) , {f}_{1} ( t ) ) , … , ( {s}_{k} ( t ) , {f}_{k} ( t ) ) ) \qquad ( 9 )$$

where OPH is the optimal policy for time horizon H and a set of Beta priors—one for each arm.

Optimal Policy Principle: For any configuration of successes and failures on all arms, the optimal policy chooses the arm with best expected reward until the end of the game.

This principle together with Property 1 and Equation (1) leads to the equations below. They compute best expected value O_{V}_{_{H}} and the optimal policy O_{P}_{_{H}} for time horizon H by dynamic programming iterating backwards from t=H down to t=0.
1) The game finishes at t=H, and there are no more rewards:

$${O}_{{V}_{H}} ( ( {s}_{1} , {f}_{1} ) , … , ( {s}_{k} , {f}_{k} ) ) = 0 , if {\sum }_{i = 1}^{k} ( {s}_{i} + {f}_{i} ) = H \qquad ( 10 )$$

2) For a discount factor d which controls the weighting or importance given to newer observed rewards as compared to older ones (for simplification of equations below, d is set to 1), the expected value V_{Hi} of pulling an arm i is given by

$${V}_{{H}_{i}} ( ( {s}_{1} , {f}_{1} ) , … , ( {s}_{k} , {f}_{k} ) ) = \frac{{α}_{i} + {s}_{i}}{{α}_{i} + {s}_{i} + {β}_{i} + {f}_{i}} ( 1 + d \cdot {O}_{{V}_{H}} ( … , ( {s}_{i} + 1 , {f}_{i} ) , … ) ) + \frac{{β}_{i} + {f}_{i}}{{α}_{i} + {s}_{i} + {β}_{i} + {f}_{i}} ( 0 + d \cdot {O}_{{V}_{H}} ( … , ( {s}_{i} , {f}_{i} + 1 ) , … ) ) . \qquad ( 11 )$$

3) For 0≤t<H (where t=Σᵢ₌₁^{k}(sᵢ+fᵢ)), the optimal strategy selects the best expected value according to the following rule:

$${O}_{{V}_{II}} ( {s}_{1} , {f}_{1} ) , … , ( {s}_{k} , {f}_{k} ) ) = {max}_{i = 1 , … , k} ( {V}_{{H}_{i}} ( ( {s}_{1} , {f}_{1} ) , … , ( {s}_{k} , {f}_{k} ) ) ) if 0 \leq {\sum }_{i = 1}^{k} ( {s}_{i} + {f}_{i} ) < H . \qquad ( 12 )$$

4) OPT selects the arm with maximal V_{Hi}

$${O}_{PM} ( ( {s}_{1} , {f}_{1} ) , … , ( {s}_{k} , {f}_{k} ) ) = {argmax}_{i = 1 , … , k} ( {V}_{{II}_{i}} ( ( {s}_{1} , {f}_{1} ) , … , ( {s}_{k} , {f}_{k} ) ) ) if 0 \leq {\sum }_{i = 1}^{k} ( {s}_{i} + {f}_{i} ) < H . \qquad ( 13 )$$

The optimal policy is organized as a one-dimensional array. In the case of 2-arm bandits, it can be indexed by the following expression:

$$Index ( ( {s}_{1} , {f}_{1} ) , ( {s}_{2} , {f}_{2} ) ) = \frac{1}{24} t ( t + 1 ) ( t + 2 ) ( t + 3 ) + \frac{1}{6} ( {s}_{1} + {f}_{1} ) ( {s}_{1} + {f}_{1} + 1 ) ( 2 {s}_{1} + 2 {f}_{1} - 3 t - 5 ) + {s}_{1} ( t - {s}_{1} - {f}_{1} + 1 ) + {s}_{2} \qquad ( 14 )$$

Most methods related to multi-armed bandits, such as the ones described above, assume that reward outcomes are received immediately after a decision is made. In most real-world scenarios, however, decision outcomes only become known some considerable delay after a decision is made. Furthermore, oftentimes multiple decisions are to be made before the outcome of a previous decision is known. The impact of such delay on existing methods and algorithms can be measured in a simulation setting.

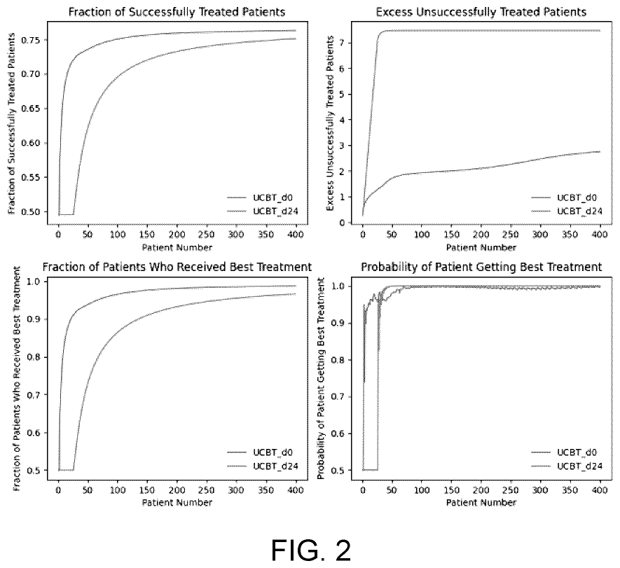

FIG. 2 highlights the manner in which a constant delay of 24 (meaning 24subsequent decisions are made before the decision reward outcome is known) compares to no delay using four different measures of algorithm performance on simulated clinical trials (Bernoulli bandits based on real data). Delayed outcomes (e.g., delayed patient treatment responses) result in a significantly smaller fraction of successfully treated patients during the trials and especially impacts the probability of successful treatment for early patients in the trial. If always providing the best treatment (77% success) to all patients is the baseline, the delay of 24 results in an additional (excess) seven unsuccessfully treated patients (83 vs. 90) over the course of the entire trial (360 patients). With no delay, the excess is just over two. Such an impact of delay is significant.

In the clinical trial example, delay has a significant impact on the effectiveness of UCBT. This was a simple example for two arms, fixed arm success probabilities, one delay value, and one algorithm. A more rigorous evaluation examining various numbers of arms is now presented. Arm priors rather than fixed success probabilities are considered and three algorithms are examined: WI, TS, and UCBT. In a single experiment, the algorithm, time horizon, number of arms and delay are pre-selected. Then arm success probabilities are drawn from their Beta priors and the algorithm plays one game making decisions solely on already known rewards.

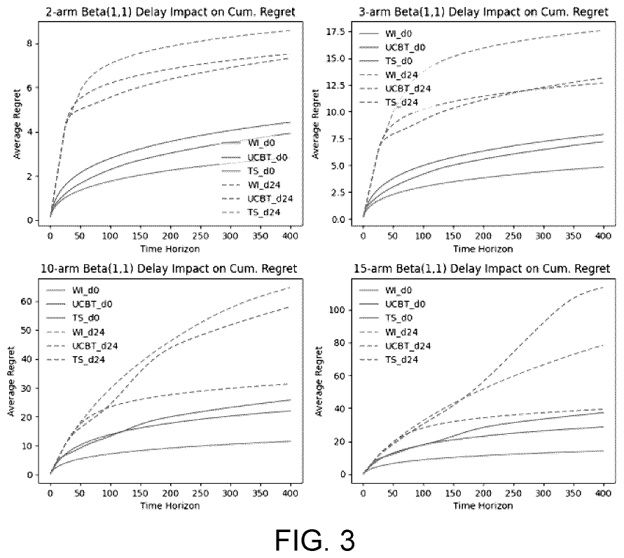

FIG. 3 presents simulation results for time horizons up to 400. Each data point is the average of one million simulation runs. From FIG. 3, it is first observed that reward outcome delays significantly deteriorate performance of all algorithms and the impact of delays increases with the number of arms. It is further observed that Thompson sampling is least sensitive to reward delays while the Whittle index, which is nearly optimal in the absence of delays, performs poorly.

The UCBT algorithm and other algorithms presented herein make decisions solely from fully observed rewards. This means that these algorithms do not take into account arm pulls which still have not returned a reward value due to delay. Unfortunately, it is not possible to use the optimal policy in such a way. The optimal policy works under the strict assumption that all rewards are always known before any subsequent decision.

As delay is such a common occurrence in the real world and it degrades common algorithm performance, methods which can improve algorithms when delay is present could be very valuable.

Starting from the principles of optimal policy computations using classic probability theory, it is possible to compute the optimal policy under delay for Bernoulli bandits, which makes optimal decisions at any stage of the game for any set of still unknown rewards. The optimal policy indexing scheme makes such computations practically feasible for 2-arm and 3-arm bandits.

The outcome analysis of Unknown-Rewards, with actual (yet unknown) arm states will now be described. A Bernoulli multi-armed bandit having an arm is considered and let (s,f) be the arm's currently observed and known successes and failures. It is assumed that the arm has been pulled an additional u times and the corresponding rewards are still unknown due to delay (there is s+f+u total arm pulls). Given that each currently unknown reward (due to delay) is either a success or a failure (property of Bernoulli multi-armed bandit), it is desirable for the arm to be in one of the following actual success/failure states: (s,f+u),(s+1,f+u−1),(s+2,f+u−2), . . . , (s+u,f), or using set-builder notation:

$$\{ ( s - δ , f + u - δ ) \vert δ ∈ ℤ and 0 \leq δ \leq u \} . \qquad ( 15 )$$

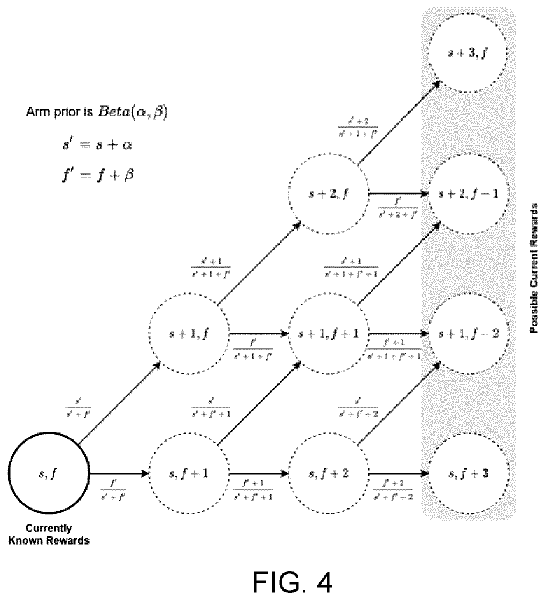

Probabilities of unknown states will now be described. FIG. 4 presents an example of all possible reward changes for u=3. This is a typical finite Markov chain model. The current observed and known reward state, by definition, has probability P[s,f)]=1. The probabilities of possible current reward states P[(s+δ,f+u−δ], 0≤δ≤u, can be computed using well-known methods. Since the expected value of a binary random variable is equal to the probability of success, state transition probabilities can be determined.

Using the notation in FIG. 4, after taking into account the arm's prior, Beta(α,β), the probability of state (s+1,f) is as follows:

$${P}^{[ ( s + 1 , f ) ] = \frac{{s}^{′}}{{s}^{′} + {f}^{′}} ℙ [ ( s , f ) ] = \frac{{s}^{′}}{{s}^{′} + {f}^{′}}} \qquad ( 16 )$$

and the probability of state (s+1,f+1) is as follows:

$$ℙ [ ( s + 1 , f + 1 ) ] = \frac{{f}^{′}}{( {s}^{′} - 1 ) + {f}^{′}} ℙ [ ( s + 1 , f ) ] - \frac{{s}^{′}}{{s}^{′} - ( {t}^{′} + 1 )} ℙ [ ( s , f - 1 ) ] . \qquad ( 17 )$$

The outcome analysis does not assume any particular sequence in which rewards are revealed to the player. It only looks at how many rewards are still unknown. This means it is valid when two different pulls of the same arm produce rewards with arbitrarily different delays.

Once currently possible rewards and their probabilities have been computed for each arm, one can examine all possible configurations of successes and failures on all arms. A single configuration is:

$$( {s}_{1} + {δ}_{1} , {f}_{1} + {u}_{1} - {δ}_{1} ) , … , ( {s}_{k} + {δ}_{k} , {f}_{k} + {u}_{k} - {δ}_{k} ) ) , \qquad ( 18 )$$

where for any arm i, 0≤i≤k, 0≤δᵢ≤uᵢ. Its probability is:

$${\prod }_{j = 1}^{k} ℙ [ ( {s}_{j} + {δ}_{j} , {f}_{j} + {u}_{j} - {δ}_{j} ) ] . \qquad ( 19 )$$

For each configuration, the expected remaining reward value of pulling arm i equals VHi (s1+δ1,f1+u1−δ1), . . . , (sk+δₖ,fₖ+uₖ−δₖ), computed using equations given previously. Consequently, taking into account all possible configurations and their probabilities, the expected remaining reward value of pulling arm i can be expressed as:

$${{\sum }^{\begin{matrix}\begin{matrix}{u}_{1} \\ …\end{matrix} \\ {u}_{k}\end{matrix}}}_{\begin{matrix}\begin{matrix}{δ}_{1} = 0 \\ …\end{matrix} \\ {δ}_{k} = 0\end{matrix}} ( {V}_{{H}_{i}} ( ( {s}_{1} + {δ}_{1} , {f}_{1} + {u}_{1} - {δ}_{1} ) , … ) {\prod }_{j = 1}^{k} ℙ [ ( {s}_{j} + {δ}_{j} , {f}_{j} + {u}_{j} - {δ}_{j} ) ] ) \qquad ( 20 )$$

where:

$${{\sum }^{\begin{matrix}\begin{matrix}{u}_{1} \\ …\end{matrix} \\ {u}_{k}\end{matrix}}}_{\begin{matrix}\begin{matrix}{δ}_{1} = 0 \\ …\end{matrix} \\ {δ}_{k} = 0\end{matrix}} ( arg ) = {\sum }_{{δ}_{1} = 0}^{{u}_{1}} ( {\sum }_{{δ}_{2} = 0}^{{u}_{2}} ( … ( {\sum }_{{δ}_{k} = 0}^{{u}_{k}} ( arg ) ) ) ) \qquad ( 21 )$$

Now the optimal policy under delay can be expressed as:

$$( 22 )$$

$${O}_{{PUD}_{H}} ( ( {s}_{1} , {f}_{1} , {u}_{1} ) , … , ( {s}_{k} , {f}_{k} , {u}_{k} ) ) = {argmax}_{i = 1 , … , k} {{\sum }^{\begin{matrix}\begin{matrix}{u}_{1} \\ …\end{matrix} \\ {u}_{k}\end{matrix}}}_{\begin{matrix}\begin{matrix}{δ}_{1} = 0 \\ …\end{matrix} \\ {δ}_{k} = 0\end{matrix}} ( {V}_{{H}_{i}} ( ( {s}_{1} + {δ}_{1} , {f}_{1} + {u}_{1} - {δ}_{1} ) , … ) {\prod }_{j = 1}^{k} ℙ [ ( {s}_{j} + {δ}_{j} , {f}_{j} + {u}_{j} - {δ}_{j} ) ] )$$

The optimal policy under delay is a generalization of the optimal policy for the classic Bernoulli bandit problem, where all rewards are known before the next decision is made. The arm with the best expected value is selected. This is, by definition, the best achievable regret performance by any algorithm.

The expression for O_{PUD}_{_{H}}((s₁,f₁,u₁), . . . , (sₖ,fₖ,uₖ)) combines elements of the classic no-delay optimal policy computations (V_{Hi}) with results of Markov chain analysis (P[(sᵢ+δᵢ,fᵢ+uᵢ−δᵢ)]). Taking into account delays adds a significant computational cost.

As can be seen in FIG. 4, the memory requirement for Markov chain computations is linear with respect to the number of unknown rewards, u. The number of floating point multiplications and divisions for one arm is ²Σᵢ₌₁^{u}2i=2(u²+u) and Markov chain computations can be parallelized for bigger values of u.

It is assumed that V_{H} is precomputed and P[(sᵢ+δᵢ,fᵢ+uᵢ−δᵢ)] are evaluated on the fly. V_{H} storage requirements for small numbers of arms and various time horizons are presented in Table 1 below.

TABLE 1
Number of floats in the precomputed part of Equation (2).

| | Time Horizon | | | | |
|---|---|---|---|---|---|
| | 100 | 200 | 300 | 400 | 1000 |
| 2-arms | 2.3M | 35.2M | 175M | 548M | 21 G |
| 3-arms | 297M | 16.8 G | 184 G | 1 T | — |

It is observed that, by using a memory indexing method, the optimal policy under delay can be used in practice for up to 3 arms.

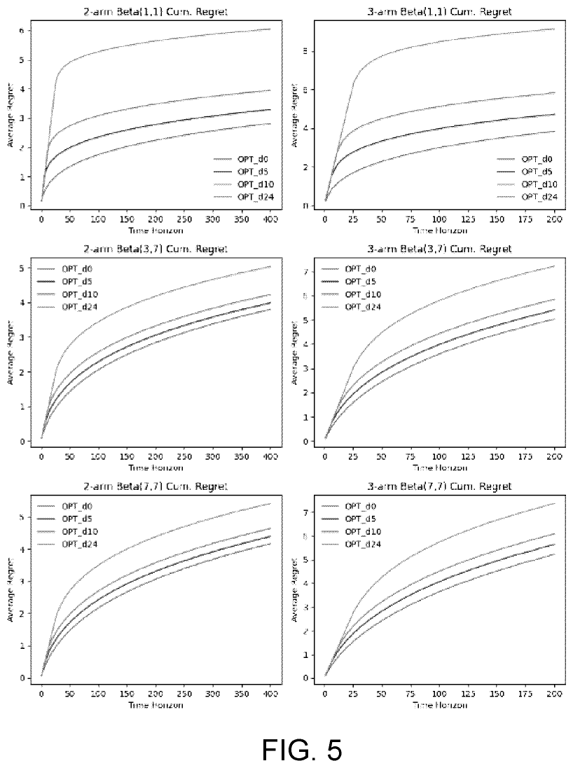

The V_{H} tables for 2-arms and 3-arms may be calculated with various priors and all time horizons up to 400 and 200, respectively. OpuDH may be evaluated under these conditions by averaging the results of one million simulation runs. Results are shown in FIG. 5. From FIG. 5, it can be seen that delays cause significant excess regret even under optimal decisions, but the optimal-policy-under-delay regret is substantially lower than the regret for suboptimal algorithms (compare FIG. 5 with FIG. 3).

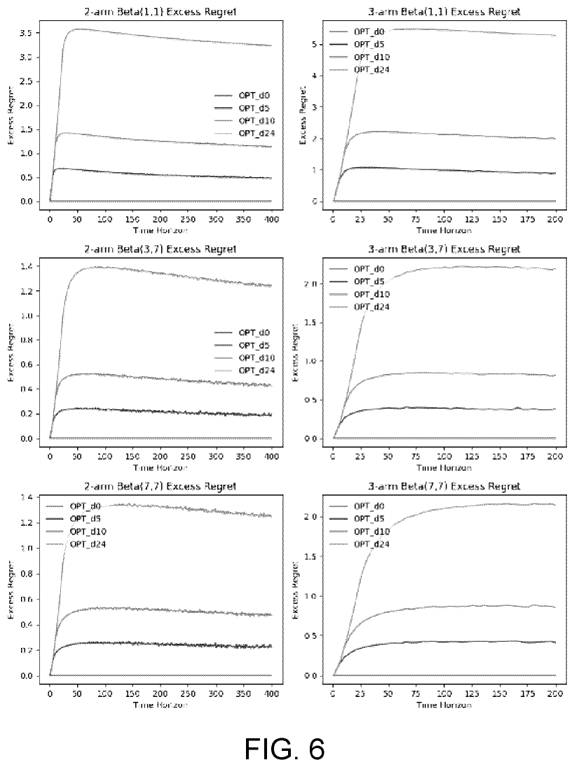

Excess regret caused by delay can be visualized by plotting the difference with the corresponding no-delay values. FIG. 6 presents such plots. From FIG. 6, it can be seen that the optimal policy's excess regret caused by delay is largest for the uniform prior, i.e., Beta(1,1), and increases with delay.

The meta-algorithm proposed herein, referred to as “Predictive Algorithm Reducing Delay Impact” or PARDI, will now be described. The optimal policy under delay becomes difficult to use efficiently in practice when the number of arms is greater than three. Computational and storage requirements become prohibitive on standard computing machines.

Every suboptimal algorithm presented beforehand, as well as other published algorithms, calculates some value measure of selecting a given arm (eval arm) and applies argmax, just like the optimal policy. This similarity suggests that the principles of the expression in Equation (2) which calculates the optimal policy under delay, O_{PUD}_{ᵤ}((s₁,f₁,u₁), . . . , (sₖ,fₖuₖ)), can be applied to algorithms other than the optimal policy. The key element of the optimal policy under delay is the consideration (prediction) of all possible-yet unknown-current rewards and their probabilities at a given time. Such probabilities are a result of existing priors and remain independent of the valuation function. Thus, the valuation part of the optimal policy under delay, V_{Hi}, can be replaced by the valuation function from any other algorithm, e.g., from UCBT. Consequently, the principles of the optimal policy under delay can be abstracted out into a meta-algorithm which takes a valuation function (eval arm) as an input. This meta-algorithm is referred to as the Predictive Algorithm Reducing Delay Impact (PARDI). The PARDI meta-algorithm is applied to the optimal policy's valuation function using mathematical notation as shown below. As previously discussed, this is the optimal policy under delay. This mathematical formulation describes PARDI for a Bernoulli bandit.

$$no - delay optimal policy : choose ( t ) = {argmax}_{i = 1 , … , k} ( ) , where$$

$$= {V}_{{II}_{i}} ( ( {s}_{i} , {f}_{i} ) , … , ( {s}_{k} , {f}_{k} ) ) \text{?}$$

$$PA RDI meta algorithm : choose ( t . ) = {argmax}_{f = 1 , … , k} ( meta\_eval {\_arm}_{i} ( ( ( {s}_{i} , {f}_{i} , {u}_{i} ) , … , ) , ) )$$

$$meta\_eval {\_arm}_{i} ( ( ( {s}_{1} , {f}_{1} , {u}_{1} ) , … ) . ) = {{\sum }_{\text{?}}}^{\text{?}} ( \text{?} )$$

$$\text{?} \text{indicates text missing or illegible when filed}$$

The method makes determining optimal decision policies for Bernoulli bandit contexts with more than three (3) arms with time horizons up to 300+and large delayed rewards possible on any suitable computing device (e.g., home personal computers). Before, such policies were considered completely computationally infeasible. Even without delayed rewards (a much easier problem), previous results were limited to three (3) arms and a time horizon of around 30. The methods and systems proposed herein therefore provide a notable technological improvement. It can be noted that the predictive approach derived for computing the optimal policy under delay can be extended to many common suboptimal algorithms, e.g., UCBT.

For most algorithms, including UCBT, TS, and WI, the meta-algorithm can be simplified as the algorithms evaluate each arm independently of others. This means that other arms do not affect the valuation of a given arm i and variables associated with them can be eliminated. Thus, in the simplified meta algorithm (referred to herein as PARDI-S), eval_armᵢ((s₁,f₁), . . . , (sₖ,fₖ)) from PARDI is replaced with a simpler eval_arm(sᵢ+δ,fᵢ+uᵢ−δ,t,H), which has probability P[(sᵢ+δ,fᵢ+uᵢ−δ)]. Consequently, as it is no longer necessary to examine all possible configurations,

$${\sum }_{\begin{matrix}\begin{matrix}{δ}_{1} = 0 \\ …\end{matrix} \\ {δ}_{k} = 0\end{matrix}}^{\begin{matrix}\begin{matrix}{u}_{1} \\ …\end{matrix} \\ {u}_{k}\end{matrix}}$$

is replaced with

$${\sum }_{δ = 0.}^{{u}_{i}}$$

The PARDI-S meta-algorithm can be applied to the Whittle index using mathematical notation as shown below.

$$no - delay algorithm : choose ( t ) = {argmax}_{i = 1 , … , k} ( ) \text{?}$$

$$PA RDI - S meta algorithm : choose ( t . ) = {argmax}_{i = 1 , … , k} ( meta\_eval \_arm ( {s}_{i} , {f}_{i} , {u}_{i} , t , H . ) )$$

$$meta\_eval {\_arm}_{i} ( {s}_{i} , {f}_{i} , {u}_{i} , t . H , ) = {\sum }_{δ = 0}^{{u}_{i}} ( \text{?} )$$

$$\text{?} \text{indicates text missing or illegible when filed}$$

It can be noted that, when arms are evaluated independently (practically by all algorithms), PARDI-S is equivalent to PARDI as it merely eliminates redundant calculations.

Still, it remains desirable to use PARDI for algorithms which do not evaluate arms independently (e.g., the optimal policy).

Algorithm 1 below presents PARDI-S in an algorithmic manner. PARDI-S keeps track of all statistics required for valuation by the applied bandit algorithm. It is desirable for PARDI-S to internally maintain and update priors as a result of successes and failures to calculate probabilities. The algorithm iterates over each arm. For each possible arm state, PARDI-S calculates the state's probability and calculates the expected valuation. Finally, it selects the arm with the largest valuation. Statistics can be kept from the very beginning of a game or from a smaller recent window of more recent observations.

For each arm, with a number of unknown rewards u, the number of floating point multiplications and divisions is ²Σᵢ₌₁^{u}2i=2(u²=u). For algorithms which cannot use the PARDI-S optimization, computational cost grows (worst case) exponentially with the number of arms. When PARDI-S is used, the total number of multiplications grows (worst case) linearly with the number of arms. As PARDI-S is a computational complexity optimization of PARDI available for some algorithms, no distinction will be made between PARDI and PARDI-S throughout the remainder of the present disclosure.

Algorithm 1: PARDI-S

Result: Elimination or reduction of delay impact
Input: Context: priors [symbol] time horizon H [symbol] time t
Input: Current (sₜ [symbol] fₜ [symbol] uₜ) for each arm i [symbol] 1 ≤ i ≤ k [symbol]
Input: eval_arm function of a bandit algorithm [symbol] e.g

[symbol]

eval_arm = WhittleIndex(s [symbol] f [symbol] H − t)

| Output: Arm with best valuation under delay | |
|---|---|
| Data: E[i] [symbol] 1 ≤ i ≤ k [symbol] arms' valuations under delay | |
| foreach arm i do | |
| \| | E[i] = 0; |
| \| | foreach state (sᵢ + δ [symbol] fᵢ + uᵢ − δ) [symbol] 0 ≤ δ ≤ uᵢ do |
| \| | \| p = [symbol] (sᵢ + δ [symbol] fᵢ + u [symbol] − δ)] : / * Markov */ |
| \| | \| e = eval_arm(s [symbol] f [symbol] H − t): |
| \| | \| E[ [symbol] = E [symbol] ] − p · e: |
| \| | end |

end
return argmaxᵢ₌ [symbol] ^{k} E [symbol] [symbol]
indicates data missing or illegible when filed

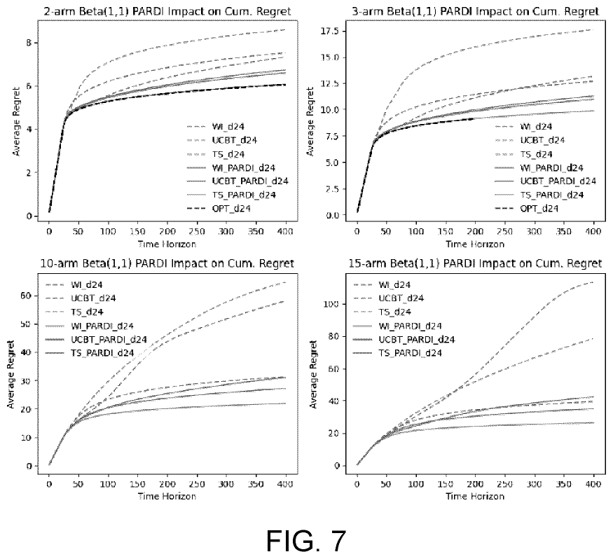

PARDI may be applied to UCBT, WI, and TS algorithms and their performance evaluated. Each algorithm is evaluated on a Bernoulli multi-armed bandit and results are averaged over a simulation study in FIG. 7. A summary of regret performance for delay of 24 can be found in FIG. 7 and Table 2 below. Each algorithm with and without PARDI is presented for 2-arms, 3-arms, 10 arms, and 15-arms with probabilities drawn from the Beta (1,1) distribution.

From FIG. 7 and Table 2, it can be noted that PARDI significantly improves the performance of tested algorithms: PARDI eliminates up to 93% of excess regret and decreased cumulative regret by up to 3×. It should be noted that applying the methodology behind PARDI in combination with the WI for scoring results in optimal (OPT)-level performance in the presence of delay, while WI alone resulted in extremely suboptimal decision-making when delay was present.

TABLE 2
PARDI: Reduction of excess-regret-due-to-delay and regret-
decrease-factor for time horizon 400; Beta (1, 1).

| | | without PARDI | | with PARDI | | |
|---|---|---|---|---|---|---|
| Alg. | arms | delay 0 | delay 24 | delay 24 | reduct. | deer. |
| WI | 2 | 2.8315 | 8.5814 | 6.0661 | 44% | 1.41× |
| | 3 | 4.8406 | 17.5802 | 9.8815 | 64% | 1.78× |
| | 10 | 11.5299 | 64.7024 | 22.0047 | 80% | 2.94× |
| | 15 | 14.1046 | 78.4476 | 26.4493 | 81% | 2.97× |
| UCBT | 2 | 3.9263 | 7.3225 | 6.7346 | 17% | 1.08× |
| | 3 | 7.2051 | 13.1538 | 11.2846 | 31% | 1.17× |
| | 10 | 25.7976 | 58.0500 | 31.1195 | 83% | 1.87× |
| | 15 | 37.3594 | 113.4912 | 42.3696 | 93% | 2.68× |
| TS | 2 | 4.4300 | 7.5226 | 6.5866 | 30% | 1.14× |
| | 3 | 7.8876 | 12.6814 | 10.9589 | 36% | 1.16× |
| | 10 | 22.0499 | 31.3632 | 27.2134 | 45% | 1.15× |
| | 15 | 28.6613 | 39.4877 | 34.9860 | 42% | 1.13× |

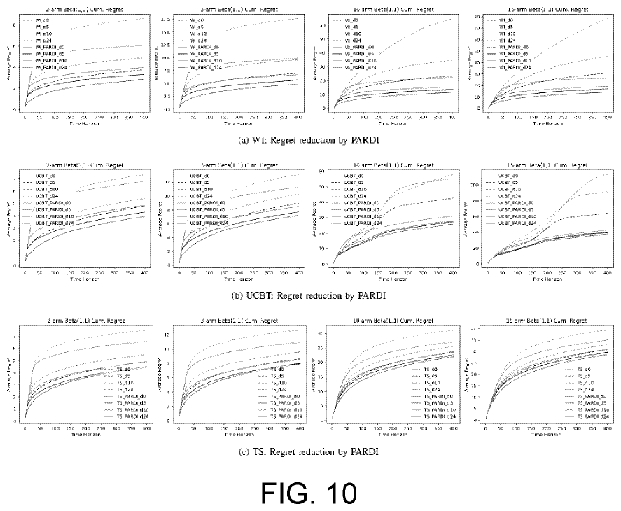

2-arm and 3-arm plots in FIG. 7 also show optimal-policy-under-delay results. They are labeled OPT_d24 and overlap plot WI-PARDI_d24. An examination of data in FIG. 5 and the corresponding data in FIG. 10(a) leads to the following observation: for 2-arm and 3-arm delayed reward bandits, WI-PARDI offers nearly optimal regret performance.

Data in Table 2 supports this claim by comparing the optimal regret to WI-PARDI regret for various delays and arm priors.

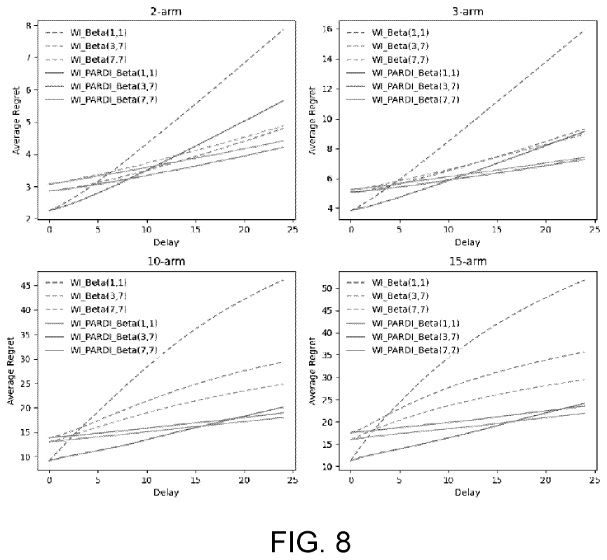

FIG. 8 shows WI regret and WI-PARDI regret as a function of delay for three different priors. WI-PARDI regret growth vs delay appears to be slightly faster than linear.

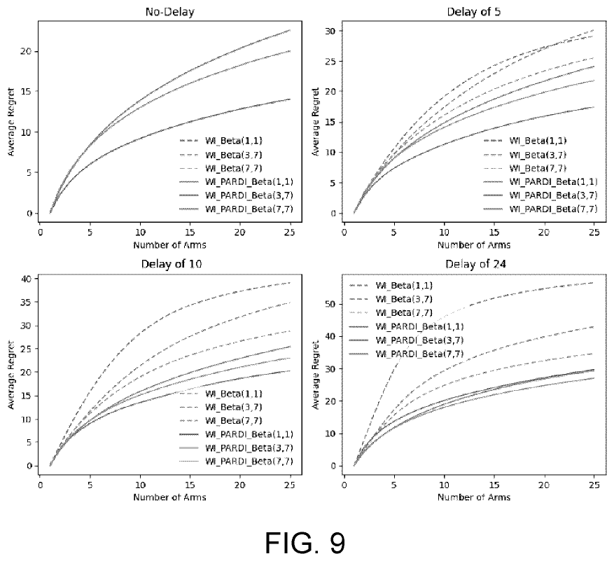

FIG. 9 shows WI regret and WI-PARDI regret as a function of the number of arms.

FIG. 10 shows regret reduction by WI-PARDI (FIG. 10(a)), UCBT-PARDI (FIG. 10(b)) and TS-PARDI (FIG. 10(c)) for various delay values and number of arms.

It can be noted that WI-PARDI regret increases with greater delays and number of arms. Varying the prior Beta distribution has a significant and complex effect. Nonetheless, WI-PARDI produces much better performance in all situations than any existing technique not utilizing the method presented herein.

While PARDI has so far been presented in the context of Bernoulli multi-armed bandits, its principles may be extended to other bandit varieties and non-binary reward distributions, e.g., via probabilistic sampling. In other words, while Bernoulli multi-armed bandits are used as an example embodiment of the methods and systems described herein, it should be understood that any suitable distribution other than the Beta distribution may apply.

Arms' priors have a dramatic impact on regret and algorithms' performance. Uniform probability distributions often assumed in theory and simulation studies are hardly applicable to real-world processes. Conclusions based on such assumptions should be reevaluated in the context of specific practical applications. Uniform probability distributions can be used, however, for qualitative analysis.

Uniform probability distributions for arm priors may be used to demonstrate the scope of delay impact on three well-known algorithms in a traditional multi-armed bandit with a Gaussian reward distribution. For each arm its N(μ,σ²) is drawn, where both μ and σ are random variables such that μ˜U(0,1) and σ˜U(a,b), b≥a≥0. Selection of a and b has a dramatic impact on simulation results.

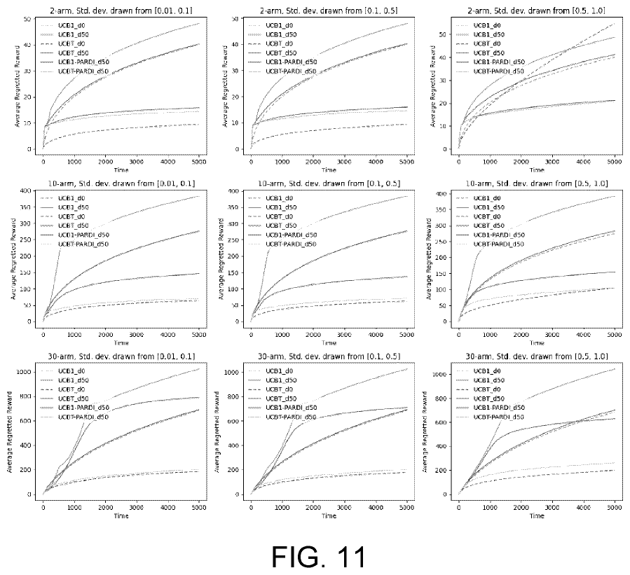

FIG. 11 presents impact of delay on regret as a function of time for bandits with Gaussian reward distributions. It shows how a constant delay of 50 affects the performance of three well-known algorithms. Three rows of sub-figures concern 2-arm, 10-arm, and 30-arm bandits respectively. For each number of arms three different ranges of arms' σ are examined. In the first column of sub-figures, σ˜U(0.01,0.1); these are situations where the arms' σ are small in comparison to the range of μ values. In the second column σ˜U(0.1,0.5); these are situations where the arms' sigma are smaller than the average μ. In the third column σ˜U(0.5,1.0); these are situations where the arms' sigma are larger than the average μ.

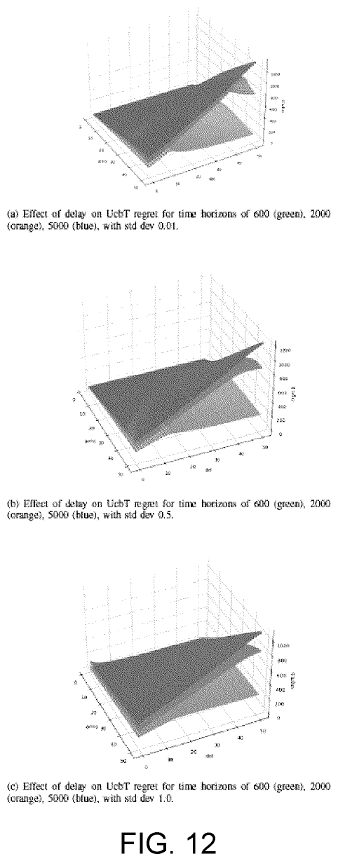

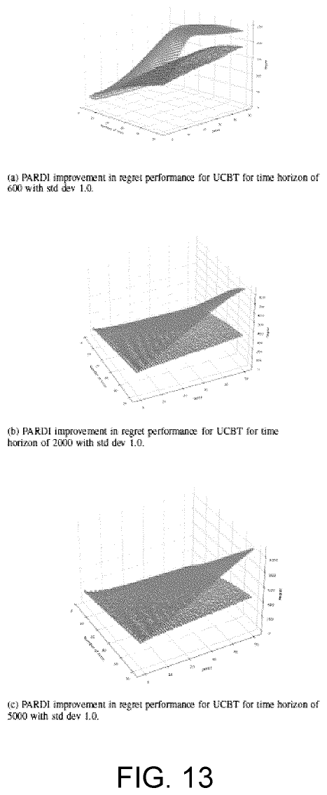

FIG. 11 and FIG. 12 show the impact of delay on existing algorithms. FIG. 11 and FIG. 13 show the reduction of delay impact by PARDI.

It can be noted that, in experiments, the performance of ϵ-greedy is significantly worse than the performance of any of the other three well-known algorithms. Consequently, plots related to e-greedy are excluded as they would only obscure other results. It is further noted that, in the absence of delays, for a particular range of bandit parameters including time, each of the three well-known algorithms can be best or significantly worse than others. In addition, in simulations, delays deteriorate the performance of UCB1. Furthermore, delays force algorithms to perform more “blind” (random) exploration. They deteriorate regret in some early stages of the game but may significantly improve regret in long runs.

Most common distributions, like the normal distribution, do not have finite reward outcome possibilities. This prevents techniques like PARDI from being traditionally applied as there are no discrete configurations with calculable probabilities. Given the demonstrated success of PARDI for Bernoulli bandits with delay, its principles may be extended to reducing the impact of delay for the normal distribution. PARDI can be modified to use probabilistic sampling or Monte Carlo sampling instead of probability measures. As nearly all algorithms evaluate each arm independently, the algorithm for this context is presented. The algorithm is run with a preset number R probabilistic sample simulations. Algorithm 2 presents this modified version of PARDI, referred to herein as PARDI-PS-S, in an algorithmic manner.

| Algorithm 2: PARDI-PS-S (Based on PARDI-S [17]) | | | |
|---|---|---|---|
| Result: Elimination or reduction of delay impact | | | |
| Input: Parameters: #arms k, time horizon H, time t | | | |
| Input: Parameters: other bandit systems' params | | | |
| Input: Context: priors [symbol] ᵢ, 1 ≤ i ≤ k. | | | |
| Input: Context: current reward history for each arm | | | |
| i, 1 ≤ i ≤ k. | | | |
| Input: eval_arm function of a bandit algorithm, e.g., | | | |
| ${eval\_arm}_{i} = {\hat{μ}}_{i} ( t ) + \sqrt{\frac{ln t}{{n}_{i} ( t )} min ( \frac{1}{4} , {V}_{i} ( t ) )} ( UCBT )$ | | | |
| Input: Number of probabilistic samples R | | | |
| Output: Arm with best sampled valuation under delay | | | |
| Data: E[i], 1 ≤ i ≤ k, arms' valuations under delay | | | |
| Data: [symbol] , probability distribution. | | | |
| foreach arm i do | | | |
| \| | [symbol] = [symbol] ᵢ. | | |
| \| | E[i] = 0 | | |
| \| | repeat R times | | |
| \| | \| | Let \hat{μ}′ᵢ = \hat{μ}ᵢ | |
| \| | \| | Let (\hat{σ}′ᵢ)² = \hat{σ}ᵢ² | |
| \| | \| | Let uᵢ = the number of yet unknown rewards | |
| \| | \| | repeat uᵢ times | |
| \| | \| | \| | Sample s from distribution [symbol] |
| \| | \| | \| | [symbol] = posterior( [symbol] , s) // See note³ |
| \| | \| | \| | Update \hat{μ}′ᵢ, (\hat{σ}′ᵢ)² and params from sample s |

| | | |
|---|---|---|
| \| | \| | end |
| \| | \| | E[i]+ = eval_armᵢ(\hat{μ}′ᵢ, \hat{σ}′ᵢ, nᵢ, t, H, params) |

```
    |  end
    | E[i] = E[i]/R
  end
  return argmaxᵢ₌₁^{k} E[i].
```

³Note:
It can be an approximation for the posterior distribution.

The above formulation can be explained with the following sequence of steps:

For each arm i:
1. Get sequence of random samples simulating the missing outcomes from previous decisions on the arm.
2. Evaluate the final decision state state of the sequence using the provided bandit algorithm (e.g., UCB1, UCBT, Thompson Sampling, EXP4).
3. Repeat steps 1 and 2 for some R (e.g., 1000 samples).
4. Determine the average of the R evaluations resulting from performing step 2 R times.
5. Select the arm i which exhibited the largest average arm evaluation from its step 4.

Applying this formulation of PARDI to algorithms such as UCBT can also result in significant performance improvements (up to 95%+ reduction in excess regret) in the presence of delay, as shown in Table 3 below. Improvement generally increases with number of arms. FIG. 13 highlights the effects of PARDI on UCBT for bandits with Gaussian reward distributions.

TABLE 3
PARDI-UCBT: Reduction of excess-regret-due-to-delay for each
arm μ~u(0, 1) and σ~u(0.1, 0.5).

| | | without PARDI | | with PARDI | |
|---|---|---|---|---|---|
| arms | time | delay 0 | delay 50 | delay 50 | reduct. |
| 2-arms | 600 | 4.56 | 11.28 | 10.56 | 10.7% |
| | 2000 | 7.08 | 13.76 | 12.47 | 19.3% |
| | 5000 | 9.45 | 16.05 | 14.28 | 26.8% |
| 10-arms | 600 | 31.12 | 78.00 | 40.32 | 80.4% |
| | 2000 | 47.60 | 113.40 | 54.80 | 89.1% |
| | 5000 | 62.01 | 137.50 | 68.50 | 91.4% |
| 30-arms | 600 | 80.40 | 153.60 | 84.00 | 95.1% |
| | 2000 | 131.80 | 588.00 | 136.00 | 99.1% |
| | 5000 | 180.00 | 700.00 | 186.50 | 98.7% |

The methodology behind PARDI does not rely on a particular technique of determining possible outcomes. Above were presented methods which enumerated all possibilities or used probabilistic samples. There can exist other methods including, but not limited to, simulation, theoretical estimates, and/or neural network(s).

In some embodiments, the methods and systems described herein are used to select at least one action from a plurality of actions to be performed in an environment including, but not limited to, when treating medical patients in medical applications, in pre-clinical and clinical trials, when routing telecommunications (e.g., to increase bandwidth), when playing games, when buying products (such as groceries), when setting prices (e.g., in dynamic pricing, or for promotional purposes), in portfolio/inventory management (e.g., for resource waste, and in A/B testing). For example, in a telecommunications environment (e.g., in a cloud computing environment), the methods and systems described herein may be used to select a resource allocation to implement (e.g., to determine a best route to use). In an Internet-of-Things (IoT), the methods and systems described herein may be used to select a channel to use from a plurality of channels. In a clinical trial or a pre-clinical trial environment, the methods and systems described herein may be used to select at least one of a drug or treatment to administer, medical equipment to use, and device option(s) to set. In an experimental (e.g., laboratory) environment, the methods and systems described herein may be used to select experimental option(s) to evaluate. In a food retail environment, the methods and systems described herein may be used to select a price at which to set one or more products, an amount of the one or more products to order, and/or a time at which to the order one or more products. It should be understood that any other suitable actions may be selected and performed in a physical environment using the methods and systems described herein.

The embodiments described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

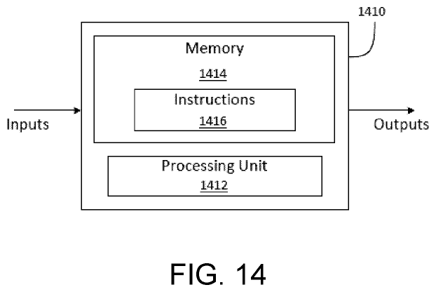

FIG. 14 is a schematic diagram of a computing device 1410, exemplary of an embodiment. As depicted, computing device 1410 includes at least one processing unit 1412, memory 1414, and program instructions 1416 stored in the memory 1414 and executable by the processing unit 1412.

For simplicity only one computing device 1410 is shown but system may include more computing devices 1410 operable by users to access remote network resources and exchange data. The computing devices 1410 may be the same or different types of devices. The computing device components may be connected in various ways including directly coupled, indirectly coupled via a network, and distributed over a wide geographic area and connected via a network (which may be referred to as “cloud computing”).

Each processing unit 1412 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 1414 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

An I/O interface may be provided to enable computing device 1410 to interconnect with one or more input devices, such as a keyboard, mouse, camera, touch screen and a microphone, or with one or more output devices such as a display screen and a speaker.

A network interface may also be provided to enable computing device 1410 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 1410 is operable to register and authenticate users (using a login, unique identifier, and password for example) prior to providing access to applications, a local network, network resources, other networks and network security devices. Computing devices 1410 may serve one user or multiple users.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps

1. A method of selecting at least one action from a plurality of actions to be performed in an environment, the method comprising:
maintaining, for each action from the plurality of actions, count data indicative of a number of times the action has been performed and a difference between the number of times the action has been performed and a number of observed resulting rewards for the action, each reward being a numeric value that measures an outcome of a given action;
determining, from the count data and a bandit score provided by a bandit model, an expected score for each action from the plurality of actions, the bandit score provided by the bandit model for a given history of performed actions and observed rewards, and the expected score determined by determining an expected value of the bandit score given a likelihood of some of the plurality of actions having unobserved pending rewards; and
selecting, from the plurality of actions and based on the expected score for each action, the at least one action to be performed in the environment.

2. The method of claim 1, wherein selecting the at least one action comprises selecting a resource allocation to implement in a telecommunications environment.

3. The method of claim 1, wherein selecting the at least one action comprises selecting at least one of a drug to administer, a treatment to provide, medical equipment to use, and device options to set in a clinical trial or a pre-clinical trial environment.

4. The method of claim 1, wherein selecting the at least one action comprises selecting an experimental option from a plurality of experimental options to evaluate in an experimental environment.

5. The method of claim 1, wherein selecting the at least one action comprises selecting a channel from a plurality of channels to use in an Internet-of-Things (IoT) environment.

6. The method of claim 1, wherein selecting the at least one action comprises selecting at least one of a price at which to set one or more products, an amount of the one or more products to order, and a time at which to the order one or more products in a food retail environment.

7. The method of claim 1, further comprising:
receiving an indication that a reward was observed in response to a selected one of the plurality of actions being performed; and
in response, updating the count data.

8. The method of claim 1, wherein maintaining the count data comprises maintaining a count of the difference between the number of times the action has been performed and the number of resulting rewards have been observed for a given action, the count being:
a windowed count that counts how many times a reward has been observed for a given action in response to the action being performed during a recent time window that includes a fixed number of most recent time steps.

9. The method of claim 1, wherein the bandit score is determined using a Whittle index.

10. The method of claim 1, wherein the bandit score is determined using an infinite time horizon algorithm.

11. The method of claim 1, wherein the expected score for each action is determined by:
determining all discrete possible reward configurations using the count data and determining probabilities of the possible reward configurations using state transition probabilities; and
multiplying a probability of each possible reward configuration by the bandit score of each action of the given history of performed actions.

12. The method of claim 1, wherein the bandit score is determined using one of probabilistic sampling and Monte Carlo simulation,
further wherein a probabilistic sample is defined as simulating a sequence of actions for missing rewards in the count data, updating a prior expected action reward distribution after each simulated action, and determining the bandit score using simulated rewards when no missing rewards remain, and
the probabilistic sampling using a number of probabilistic samples to create an estimate of the expected score.

13. The method of claim 1, wherein the rewards measure discrete outcomes.

14. The method of claim 1, wherein the rewards measure an uncountable set of outcomes defining a continuous probability distribution.

15. The method of claim 1, wherein the expected score is an average expected score based on a number N of samples, and further wherein selecting the at least one action to be performed in the environment comprises selecting a highest average expected score.

16. A system comprising one or more computers and one or more storage devices storing instructions that when executed by the one or more computers cause the one or more computers to perform operations for selecting at least one action from a plurality of actions to be performed in an environment, the operations comprising:
maintaining, for each action from the plurality of actions, count data indicative of a number of times the action has been performed and a difference between the number of times the action has been performed and a number of observed resulting rewards for the action, each reward being a numeric value that measures an outcome of a given action;
determining, from the count data and a bandit score provided by a bandit model, an expected score for each action from the plurality of actions, the bandit score provided by the bandit model for a given history of performed actions and observed rewards, and the expected score determined by determining an expected value of the bandit score given a likelihood of some of the plurality of actions having unobserved pending rewards; and
selecting, from the plurality of actions and based on the expected score for each action, the at least one action to be performed in the environment.

17. The system of claim 16, wherein the operations further comprise:
receiving an indication that a reward was observed in response to a selected one of the plurality of actions being performed; and
in response, updating the count data.

18. The system of claim 16, wherein maintaining the count data comprises maintaining a count of the difference between the number of times the action has been performed and the number of resulting rewards have been observed for a given action, the count being:
a windowed count that counts how many times a reward has been observed for a given action in response to the action being performed during a recent time window that includes a fixed number of most recent time steps.

19. The system of claim 16, wherein the expected score for each action is determined by:
determining all discrete possible reward configurations using the count data and determining probabilities of the possible reward configurations using state transition probabilities; and
multiplying a probability of each possible reward configuration by the bandit score of each action of the given history of performed actions.

20. The system of claim 16, wherein the bandit score is determined using one of probabilistic sampling and Monte Carlo simulation,
further wherein a probabilistic sample is defined as simulating a sequence of actions for missing rewards in the count data, updating a prior expected action reward distribution after each simulated action, and determining the bandit score using simulated rewards when no missing rewards remain, and
the probabilistic sampling using a number of probabilistic samples to create an estimate of the expected score.
